(19) United States
(12) Patent Application Publication
(10) Pub. No.: US 2010/0189222 A1
(43) Pub. Date: Jul. 29, 2010

(54) PANORAMIC IRRADIATION SYSTEM USING FLAT PANEL X-RAY SOURCES

(75) Inventors: Mark Eaton, Austin, TX (US); Mitali More, Austin, TX (US); Mike Olla, Austin, TX (US)

Correspondence Address: HULSEY IP INTELLECTUAL PROPERTY LAWYERS, P.C., 919 Congress Avenue, Suite 919, AUSTIN, TX, 78701, US

(73) Assignee: Steller Micro Devices

(21) Appl. No.: 12/692,495

(22) Filed: Jan. 22, 2010

Related U.S. Application Data
(63) Continuation-in-part of application No. 12/201,741, filed on Aug. 29, 2008, now abandoned.
Application No. 12/201,741 is a continuation of application No. 11/355,692, filed on Feb. 16, 2006, now abandoned.
(60) Provisional application No. 61/249,087, filed on Oct. 6, 2009.

(51) Int. Cl. G21K 5/08

(52) U.S. Cl. 378/68

(57) ABSTRACT

The present disclosure describes a panoramic irradiator comprising at least one X-ray source inside a shielded enclosure, the one or more sources each operable to emit X-ray flux across an area substantially equal to the proximate facing surface area of material placed inside the enclosure to be irradiated. The irradiator may have multiple flat panel X-ray sources disposed, designed or operated so as to provide uniform flux to the material being irradiated. The advantages of the irradiator of the present disclosure include compactness, uniform flux doses, simplified thermal management, efficient shielding and safety, the ability to operate at high power levels for sustained periods and high throughput.

### REFERENCES TO RELATED APPLICATIONS

The present U.S. Utility Patent Application claims priority pursuant to 35 U.S.C. §120, as a continuation-in-part (CIP), to the following U.S. Utility Patent Application which is hereby incorporated herein by reference in its entirety and made part of the present U.S. Utility Patent Application for all purposes:

1. U.S. Utility application Ser. No. 12/201,741, entitled “COMPACT RADIATION SOURCE,” (Attorney Docket No. STRY002US1), filed Aug. 29, 2008, pending, which claims priority pursuant to 35 U.S.C. §120 as a continuation to the following U.S. Patent Application which is hereby incorporated herein by reference in its entirety and made part of the present U.S. Utility Patent Application for all purposes:

a. U.S. Utility application Ser. No. 11/355,692, entitled “COMPACT RADIATION SOURCE,” (Attorney Docket No. STRY002US0), filed Feb. 16, 2006, abandoned.

The present U.S. Utility Patent Application also claims priority pursuant to 35 U.S.C. §119(e) to the following U.S. Provisional Patent Application which is hereby incorporated herein by reference in its entirety and made part of the present U.S. Utility Patent Application for all purposes:

1. U.S. Provisional Application Ser. No. 61/249,087, entitled “PANORAMIC IRRADIATION SYSTEM USING FLAT PANEL X-RAY SOURCES,” (Attorney Docket No. STRY007US0), filed Oct. 6, 2009, pending.

### GOVERNMENT INTEREST

This invention was made with Government support under Contract No. 70NANB7H7030 awarded by the Advanced Technology Program of the National Institute of Standards and Technology. The U.S. Government has certain rights in the invention.

### TECHNICAL FIELD OF THE INVENTION

The present disclosure relates generally to an irradiation system and method, and more particularly, to a panoramic X-ray irradiator system and method wherein the X-ray flux generation area of a source is substantially equal to the proximate target surface area of material passing through the irradiator.

### BACKGROUND OF THE INVENTION

Ionizing radiation, such as electron beams, gamma rays and X-rays, is widely used for the irradiation treatment of objects, including: the sterilization of medical, pharmaceutical, food and cosmetic products; the cross-linking of polymers and other industrial processes; the inactivation of leukocytes in transfusion blood supplies; the sterilization of insects for phytosanitary; the attenuation of organism function for vaccine development, and many other applications.

Broadly speaking, irradiators are classified as either self-contained irradiators or panoramic irradiators. In self-contained irradiators, the radiation source, radiation shielding, the objects to be treated, any systems for the movement of those objects, and sometimes the power supply, are all in one enclosure. X-ray versions are regulated by the U.S. Food and Drug Administration under the category “X-ray cabinet irradiator” (Title 21 CFR §1020.40). Panoramic irradiators are generally larger than the self-contained irradiators and use a material transport system to move the materials to be treated from an area where people may safely operate to a separately-shielded irradiation area receiving flux from the radiation source. They are most commonly used for the irradiation of large volumes of material.

The radiation source used in either type of irradiator may include: gamma rays emitted by the decay of radioactive isotopes; electron beams produced by linear accelerators, electron tubes or other methods; or X-rays produced by the impact of high energy electrons upon a metal target, for example in an X-ray tube.

The predominant radiation sources for panoramic irradiators are radioactive isotopes and electron beams (e-beams). Both emit very high energy radiation of over 1 MV to as much as 10 MV and thus require massive metal and concrete shielding to protect workers at these facilities and surrounding populations. Panoramic irradiator facilities have a separate, shielded area in which workers can safely load the material to be irradiated onto a material transport system which delivers the material into the irradiation area, where it can either remain stationary or be moved at a regulated pace for as long as is required for the desired dose of radiation to be delivered. Typical materials processed at these facilities are packaged medical products, which are then not exposed to an outside environment or human handling until the package is opened at the point of use, mail or packages being shipped, and some foodstuffs. The doses delivered to these materials are generally much higher than those delivered to materials in self-contained irradiators. For example, foodstuffs can require doses of a few hundred Gy to a few kGy in order to sterilize the bacteria, mold or yeasts which are commonly of concern for food safety. Medical products typically require 15 kGy to 25 kGy in order to sterilize bacteria, mold, yeasts, mold and bacterial spores, viruses and prions which are of concern in medical product safety. Radioactive isotope panoramic irradiators commonly use Cobalt-60, emitting mostly 1.25 MV photon flux, which is formed into rods. The rods line the perimeter of the irradiation area, which is commonly a pit dug into the ground for additional shielding. Material to be irradiated is loaded into large “totes”, commonly of 650 KG mass, in the safe loading area. These totes are then moved by hook and cable or other material conveyance apparatus into the radiation pit, where they remain until the required dose is delivered. The material is then removed from the pit and transferred by the material conveyance system to an unloading area. These facilities are large and centralized to serve regional markets. There are under 100 of them in the United States. Placing the isotope rods in the radiation area and removing them once they have decayed is extremely hazardous and requires the use of remotely controlled equipment. Co-60 is also of concern for possible use in a radioactive dispersal device (“dirty bomb”) and accounts for nearly all the radioactive activity of all isotopes used in the U.S. [US NRC 2007].

E-beam irradiators do not rely on radioactivity but instead use very high energy (typically 5 to 10 MV) e-beams generated by large electrical sources such as linear accelerators or rhodotrons. They are used for irradiation processing of some of the same materials as the isotope irradiators. These electrical sources can be turned off, which stops generation of the e-beam flux, but e-beams have the disadvantage of less penetrating ability compared with gamma ray or X-ray photons. This limits the mass of material that can be processed with these facilities, and hence their economical throughput rates, so they are less common than the isotope irradiators. Some e-beam facilities also have metal X-ray targets, the back sides of which are scanned by the e-beam source in order to generate high energy X-ray flux out the other side of the target, which is generally under 1 cm thick. These X-rays have greater penetrating ability than the e-beams which generated them, so they can be used for thicker materials. Both the e-beams and the X-rays have very high energies, which requires the radiation area to be heavily shielded with metal and concrete. Material is commonly loaded onto conveyor belts in a separate area and then transported into the radiation area. These facilities are also large and centralized to serve regional markets.

E-beam irradiators do not rely on radioactivity but instead use very high energy (typically 5 to 10 MV) e-beams generated by large electrical sources such as linear accelerators or rhodotrons. They are used for irradiation processing of some of the same materials as the isotope irradiators. These electrical sources can be turned off, which stops generation of the e-beam flux, but e-beams have the disadvantage of less penetrating ability compared with gamma ray or X-ray photons. This limits the mass of material that can be processed with these facilities, and hence their economical throughput rates, so they are less common than the isotope irradiators. Some e-beam facilities also have metal X-ray targets, the back sides of which are scanned by the e-beam source in order to generate high energy X-ray flux out the other side of the target, which is generally under 1 cm thick. These X-rays have greater penetrating ability than the e-beams which generated them, so they can be used for thicker materials. Both the e-beams and the X-rays have very high energies, which requires the radiation area to be heavily shielded with metal and concrete. Material is commonly loaded onto conveyor belts in a separate area and then transported into the radiation area. These facilities are also large and centralized to serve regional markets.

The massive shielding needed to protect of people from very high energy radiation adds substantially to the cost of these prior art panoramic irradiators. The need for producers to ship their product material to centralized radiation processing facilities, where the material must then be handled several extra times, adds substantially to the incremental costs of the product. The time spent shipping product to and from the panoramic irradiator facilities and the time spent during the irradiation operation add substantially to the inventory costs of producers. As a result of these added costs in time and money, many materials which might be sterilized with radiation are either not sterilized at all, as is the case with many foodstuffs and mail, or are sterilized using other techniques, such as some medical products now sterilized with ethylene oxide, which has carcinogenic properties.

A smaller form factor and more economical panoramic irradiator using radiation flux with substantially lower energies and requiring much less shielding than prior art irradiators is desirable. Such an irradiator would not be limited to centralized locations, but could instead be used close to the point of production, the point of loading or transshipment or the point of consumption of the material to be irradiated, thereby saving substantial costs in time and money and enabling the more widespread application of beneficial radiation.

The most common prior art X-ray sources, X-ray tubes, generate flux with e-beams having energies under 200 kV and mean X-ray flux under 50 kV, with the higher energy e-beams unable to escape the vacuum tube, so shielding requirements are very relaxed compared to prior art panoramic irradiator sources.

FIG. 1 shows the general architecture of prior art X-ray tubes. X-ray tubes are point sources of radiation, as shown in FIG. 1, wherein X-rays are generated by the impact of a high voltage electron beam 50 from a heated filament or other cathode 10 at a point (sometimes called the spot) on a metal anode 30, typically disposed at an angle relative to the cathode so as to allow X-ray flux 60 to exit one side of the vacuum tube enclosing the cathode and anode. This entire side may comprise the flux exit window of the tube, or a separate window 20 of a low Z material such as beryllium may be built into this side of the tube or housing for the tube. In tubes operating below cathode to anode voltages of 150 KV, less than 2% of the energy from the electrons is converted into X-rays, while the rest is dissipated as heat on the anode.

Several limitations of X-ray tubes make them unsuitable for use in panoramic irradiators. X-ray tubes will deliver an uneven dose to the irradiation target, for example a blood bag, since the X-rays will first impinge on one surface of the target and then be attenuated as they pass through the target material and because the X-ray flux delivered from a point source will be weaker at the sides of the target coverage area than at the center. X-rays from a single point on the anode will be emitted in all directions. Those which go back into the target will not be useful for irradiation, but will instead generate heat. With the X-ray target angled as shown in FIG. 1, even more of the X-rays are absorbed in the target than would be the case with a target disposed normal to the axis of the electron beam, a phenomenon known as the heel effect. Irradiation efficiency is further reduced by the fact that, of those X-rays directed away from the target, only those which impinge on the irradiation target surface will do useful work; the rest are absorbed by shielding structures. At the same time, the target surface area, to be useful in most irradiation applications, must be many times larger than the spot on the anode of an X-ray tube. As the intensity of the X-ray flux is inversely related to the square of the separation, the tube output has to be increased to meet the irradiation needs.

FIG. 2 shows the throw distance needed for prior art point sources used in irradiation. The cabinet and shielding must also be enlarged to accommodate the throw distance 200 shown in FIG. 2 that is required to cover a target area 400 with length and width 410. Furthermore, since all the flux needed for the application must come from one spot on the anode, there is a tremendous thermal load on this small area, which in turn necessitates the use of complex liquid cooling systems for higher flux applications.

Multiple X-ray tubes will be not provide efficient or economical panoramic irradiation. Some recent inventions have taught the use of two or more X-ray tubes in self-contained irradiators, such as U.S. Pat. Nos. 6,212,255 and 6,614,876. The X-ray tubes used in commercial versions have been high-power models designed for applications such as computed tomography systems. Transfusion blood irradiators with two X-ray tubes have been made in which externally-connected liquid cooling systems are provided to dissipate the heat from the spots on the anodes. In practice, these irradiators have proven to be cumbersome and unreliable, thereby limiting the adoption of X-ray systems for blood irradiation [Dodd, 2009]. The dose required for transfusion blood irradiation is only 25 Gy, whereas the doses for medical product sterilization, such as is practiced in panoramic irradiators, can be as high as 25 kGy, so it will be appreciated that even a very large number of X-ray tubes would be insufficient for panoramic irradiation applications owing to thermal management limitations, apart from the cost and impracticality of using a very large number of tubes.

More recently, a new type of specimen and blood irradiator consisting of a center-filament X-ray tube that irradiates 360 degrees around the tube and a cylindrical gold target has been described in U.S. Pat. No. 7,346,147. The electron source is a thermal cathode in the form of an elongated filament mounted along the axis of the cylindrically shaped transmissive type anode. Instead of a point source as is the case in most X-ray tubes, this invention is in the form of a line source. The electrons impinge on the interior surface of the anode and the X-rays generated penetrate the anode material and exit out of the exterior surface of the anode. The anode has to be made very thin (14 micron Au on 4 mil Al) in order to generate the forward directed X-rays. Flat panel versions of this kind of source using a transmissive anode are disclosed in U.S. Pat. Nos. 6,477,233 and 6,674,837. Two major limitations of this kind of source are the thermal loading capacity of the thin-film anode, and the thermal matching of the anode to the exit window of the source. Even with externally-connected liquid cooling systems, only limited amounts of X-ray power can be obtained from this kind of source. The X-ray irradiation apparatus taught by Avnery in U.S. Pat. Nos. 6,738,451, 7,133,493, and 7,324,630 also uses X-ray sources relying on a transmissive anode/X-ray target and thus having these same limitations.

Another X-ray source had been disclosed in U.S. Pat. No. 7,447,298 having a thermionic or cold cathode array inside a vacuum enclosure, which can direct e-beam current to a thin film X-ray target disposed on an exit window located above the cathode array with reference to the direction of the e-beam and X-ray fluxes, or, with a second cathode array, to a wide area anode located below the first cathode array, the second cathode arrays and the exit window with the thin-film anode. This source will have the heat dissipation limitations as discussed above for the thin-film X-ray target. X-rays produced by the lower, “reflective” anode will be attenuated first by the cathode arrays and their support structures, and then the thin-film X-ray target, resulting in an inefficient system. The second anode, while it can be thicker and have higher heat dissipation capacity than a thin-film anode, is inside the vacuum enclosure. The heat must therefore be transferred through the vacuum enclosure, which will limit the amount of X-ray flux that can be achieved with this source.

### BRIEF SUMMARY OF THE INVENTION

Embodiments of the present disclosure are directed to systems and methods that are further described in the following description and claims. Advantages and features of embodiments of the present disclosure may become apparent from the description, accompanying drawings and claims.

Embodiments of the present disclosure provide an irradiation system and method wherein the X-ray flux generation area of a substantially planar source is substantially equal to the proximate target surface area facing X-ray flux generation area of the materials passing through the irradiator. The system utilizes one or more substantially planar X-ray source(s), which generates high intensity X-ray flux over a large area. As this X-ray flux generation area is substantially planar, the X-ray flux remains substantially uniform within the irradiation chamber. One or more flat panel X-ray sources are placed around the irradiation chamber to generate X-ray flux. The design of the present disclosure provides a compact, efficient and safe irradiation system.

Embodiments of the present disclosure provide a safe, economical and efficient panoramic X-ray irradiation system that offers significant advantages over prior art approaches. More specifically, present disclosure provides a system for X-ray irradiation wherein the X-ray flux generation area of a source is substantially equal to the proximate facing surface area of the material as it is transported through the irradiation section of the irradiator. The irradiator includes one or more flat panel X-ray source(s) which generate a wide source of X-ray flux, disposed inside a radiation shielding enclosure, with a material transport system provided to move the material to be irradiated from outside the enclosure to an irradiation section inside the enclosure. Shielded sections of the enclosure before and after the irradiation section protect surrounding people from any stray radiation. The one or more flat panel X-ray sources are disposed in the irradiation section so as to have their flux emitting surfaces facing inwards towards the material being transported through that section. With flat panel X-ray sources on either side of the material, most X-ray flux which passes by or through the material being irradiated will be absorbed by the anode of the opposite flat panel X-ray source, providing a degree of self-shielding. This and the much lower energies generated from the X-ray sources (mean energies generally under 100 kV) very substantially reduce the need for additional shielding materials as compared with prior art panoramic irradiators, one factor allowing the irradiator of this disclosure to be made in a relatively compact format. Since the X-ray sources are wide, and the flux generation area is substantially equal to the irradiation target area, minimal throw distance is needed compared with a point source, another factor allowing the irradiator to be made more compact. The irradiator of this disclosure can be made small enough to fit in the shipping bay of a product manufacturing site, to be installed in-line with a manufacturing process, or be loaded onto or assembled into a trailer. It can also be made modular, with sections of the irradiator section joined together for additional irradiation process capacity. Many types of material transport mechanisms can be used. A conveyor belt can transport solids, including packaged products. Pipes can transport fluids. Sheets of material can be transported through on rollers. The material transport mechanism provides uniform flux delivery in one dimension. The flat panel X-ray sources can be designed to provide uniformity in the second dimension. The configuration of the material being irradiated and the use of X-ray sources on multiple sides of the irradiation chamber can provide a more uniform flux dose map in the third dimension.

According to one embodiment of the present disclosure an apparatus and method for the X-ray irradiation of materials. This apparatus includes an irradiation chamber, a number of flat electromagnetic (X-ray) sources, a transport and support mechanism, a heat transfer system, and a shielding system. The transport system allows materials to be transported to and from an interior volume of the irradiation chamber. End covers provide shielding such that essentially all the electromagnetic flux remains within the irradiation system without irradiating the exterior environment. A shielded portal within the shielding system allows access to an interior volume of the irradiation chamber. The shielded portal allows materials to be placed in and withdrawn from the irradiation chamber. When closed, the shielded portal allows a continuous shielded boundary of the interior volume of the irradiation chamber. The electromagnetic sources are positioned on or embedded with interior surfaces of the irradiation chamber. These electromagnetic sources may generate an electromagnetic flux, such as an X-ray flux, where this flux is used to irradiate the interior volume of the irradiation chamber and any materials placed therein. The materials placed within the interior of the chamber may be supported by a low attenuation support mechanism. This low attenuation support mechanism does not substantially reduce the X-ray flux intended to irradiate the materials placed within the interior volume of the irradiation chamber. Additionally the irradiation chamber may have a heat transfer system thermally coupled to the irradiation chamber and electromagnetic sources in order to remove heat from the interior surfaces of the irradiation chamber. The shielding system and end covers external to the irradiation chamber prevents unwanted radiation from escaping from within the irradiation chamber.

Another embodiment of the present disclosure provides a method for the X-ray irradiation of materials. This method involves transporting a work piece or material to be irradiated to and from an irradiation chamber. The work piece or materials are placed within the irradiation chamber and supported with a mechanism such as a low attenuation support mechanism. This low attenuation support mechanism does not substantially reduce the electromagnetic flux (X-ray) flux within the irradiation chamber. One or more flat electromagnetic (X-ray) sources may be energized to irradiate the interior volume of the irradiation chamber. This allows the work piece or materials to be irradiated within the chamber. Excess heat may be removed with a heat transfer system in order to prevent the irradiation chamber/electromagnetic source from overheating. Additionally the irradiation chamber may be shielded to prevent the irradiation of objects and materials external to the irradiation chamber.

Yet another embodiment of the present disclosure provides another system for the X-ray irradiation of materials. This system includes an irradiation chamber, a number of flat X-ray sources, a transport mechanism, a low attenuation support mechanism, a heat transfer system, a shielding system, and a process controller. The irradiation chamber has an inner volume wherein the flat X-ray sources are positioned within or on the interior surfaces of the irradiation chamber such that the flat X-ray sources may irradiate the interior volume of the irradiation chamber. The transport mechanism allows materials to travel to and from the irradiation chamber. Within the irradiation chamber the low attenuation support mechanism supports the work pieces or materials to be irradiated while not substantially reducing the X-ray flux available for the irradiation of these objects. The heat transfer system removes heat from the X-ray source and the shielding system external to the irradiation chamber prevents inadvertent irradiation of materials and objects outside the irradiation chamber. The process controller coordinates the operation of the irradiation chamber, X-ray source, heat transfer system and an interlock system which prevents irradiation while access to the interior volume is open.

### BRIEF DESCRIPTION OF THE SEVERAL VIEWS OF THE DRAWINGS

For a more complete understanding of the present disclosure and the advantages thereof, reference is now made to the following description taken in conjunction with the accompanying drawings in which like reference numerals indicate like features and wherein:

FIG. 1 shows the general architecture of prior art X-ray tubes;

FIG. 2 shows the throw distance needed for prior art point sources used in irradiation;

FIG. 3 is a diagram that depicts one advantage of the irradiator provided by embodiments of the present disclosure, where the flux generation area of the source is substantially equal to the proximate facing surface area of the material being;

FIG. 4 is a diagram of the general architecture of an irradiator in accordance with embodiments of the present disclosure;

FIG. 5 is another diagram of the general architecture of flat panel X-ray sources in accordance with embodiments of the present disclosure;

FIG. 6 is a diagram of the X-ray flux distribution from two flat panel X-ray source provided in accordance with embodiments of the present disclosure;

FIG. 7 is a diagram of another embodiment of an irradiator in accordance with embodiments of the present disclosure;

FIGS. 8A and 8B shows calculated dose-depth maps of X-ray flux delivered to material in an irradiator of the present disclosure having flat panel X-ray sources placed on opposite sides of the material;

FIG. 9 shows an embodiment of the present disclosure in which the cathodes in the array of a flat panel X-ray source are made more dense towards the edges of the array away from the center, thereby smoothing out the flux distribution of the source across its emitting area;

FIG. 10 shows an embodiment of the present disclosure in which the cathodes of the array in a flat panel X-ray source are supplied with greater current the further the cathodes are away from the center of the array and towards the edges of the array, thereby smoothing out the flux distribution of the source across its emitting area;

FIG. 11 is a diagram of a fluid transportation system for in accordance with embodiments of the present disclosure; and

FIG. 12 is a diagram of a sheet roller transport system for use in accordance with embodiments of the present disclosure; and

FIG. 13 provides a logic flow diagram of a method of irradiating materials in accordance with embodiments of the present disclosure.

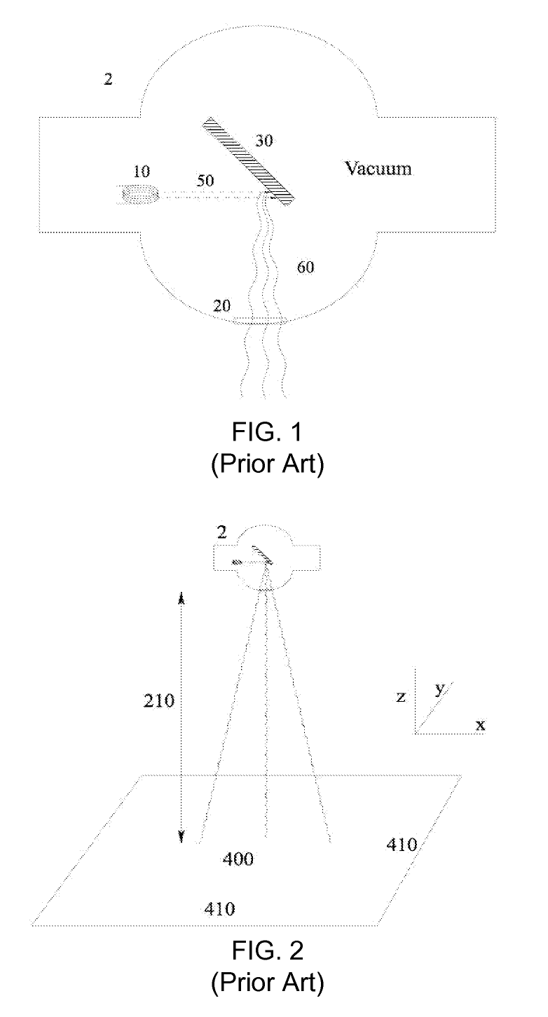

### DETAILED DESCRIPTION OF THE INVENTION

Preferred embodiments of the present disclosure are illustrated in the FIGs., like numerals being used to refer to like and corresponding parts of the various drawings.

Embodiments of the present disclosure provide an apparatus and method for the X-ray irradiation of materials. This apparatus includes an irradiation chamber, a number of flat electromagnetic (X-ray) sources, a support mechanism, a heat transfer system, and a shielding system. A shielded portal within the shielding system allows access to an interior volume of the irradiation chamber. The shielded portal allows materials to be placed in and withdrawn from the irradiation chamber. When closed, the shielded portal allows a continuous shielded boundary of the interior volume of the irradiation chamber. The electromagnetic sources are positioned on or embedded with interior surfaces of the irradiation chamber. These electromagnetic sources may generate an electromagnetic flux, such as an X-ray flux, where this flux is used to irradiate the interior volume of the irradiation chamber and any materials placed therein. The materials placed within the interior of the chamber may be supported by a low attenuation support mechanism. This low attenuation support mechanism does not substantially reduce the X-ray flux intended to irradiate the materials placed within the interior volume of the irradiation chamber. Additionally the irradiation chamber may have a heat transfer system thermally coupled to the irradiation chamber and electromagnetic sources in order to remove heat from the interior surfaces of the irradiation chamber. The shielding system external to the irradiation chamber prevents unwanted radiation from escaping from within the irradiation chamber.

Embodiments of the present disclosure improve upon prior art panoramic irradiators through the use of one or more flat-panel, broad-area X-ray sources capable of delivering more substantial flux dose rates in a format well-suited to efficient irradiation. The most general aspect of the present disclosure is the generation of the X-ray flux in the self-contained irradiator from a broad area anode, including a broad area anode that can be easily cooled to dissipate the heat produced in X-ray generation.

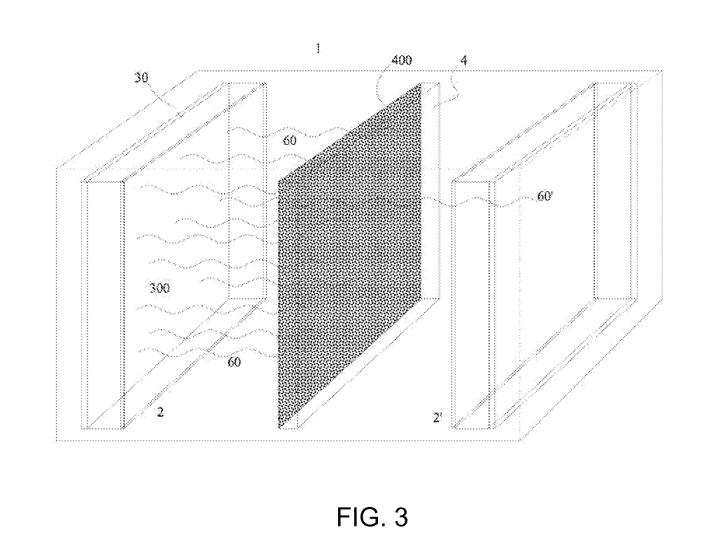

FIG. 3 is a diagram of the general architecture of the irradiator with the flux generation area of a source substantially equal to the proximate facing surface area of the material being irradiated in accordance with embodiments of the present disclosure. In self-contained irradiator 1, the flux generation area 300 on the surface of wide, flat anode 30 of a flat panel X-ray source is substantially equal to the proximate facing surface area 400 of the material to be irradiated 4, both of which are enclosed in cabinet 5 of the irradiator. Flat panel X-ray source 2 may be made in any area format, for example, circular, rectangular or square, and in sizes ranging from a few square centimeters to a square meter or more. Since flux generation area 300 and irradiation target area 400 are substantially the same, no extra throw distance is needed in the z-axis, so the X-ray sources may be placed in close proximity to the irradiation target material, allowing the irradiator to be made compact.

Among the many materials that can be irradiated in accordance with this disclosure are medical products such as sutures, bandages, surgical staples and medications; contacts lenses; pharmaceuticals; polymers being cross-linked; industrial cutting fluids; cosmetics; foodstuffs; mail and packages; water and wastewater; and vaccines. Some items may be irradiated directly, while others, such as fluids, will be irradiated inside a container or pipe penetrable by X-ray flux. Embodiments of the present disclosure are well-suited for the irradiation of materials with contoured or irregular surfaces, since X-ray flux is emitted at all angles at a multitude of locations across source anode surface 300, allowing the flux to hit the target surface from many different directions, and since source 2 may be operated at high voltage and high power to generate X-rays with high penetrating ability.

FIG. 3 also shows the use of two flat panel X-ray sources, with source 2 on one side and source 2′ on the other side of target material 4, which provides for more even distribution of X-ray flux through the material in the z direction. In other aspects of the disclosure, the sources may be oriented above and below the material, or sources may be placed on all sides of the irradiation section. The rectangular prism irradiator shown in FIG. 3 is an exemplary design; irradiator 1 may be made in circular, hexagonal, octagonal or other shapes. Flat panel X-ray sources 2 may also be designed or operated to produce different power levels or X-ray energy distributions to suit a particular application. A collimating grid may be placed in front of flat panel X-ray source 2, so as to allow the source to be used for imaging applications, with film or other X-ray detector means placed on the opposite side of material 4 from source 2.

An important advantage of multiple sources as used in the present disclosure is self shielding. Sources 2 can be operated with electrical current and anode potential calibrated to deliver as much of the generated X-ray flux as possible into the material to be irradiated. As depicted in FIG. 3, however, some of the X-rays 60′ will pass through the material and exit the opposite side. These X-rays will then be absorbed, primarily by the anode of the opposite source 2′, thereby reducing the need for additional shielding material in the irradiator. With more than two sources, for instance four sources in two opposing pairs, even more of the unused flux will be absorbed through self-shielding.

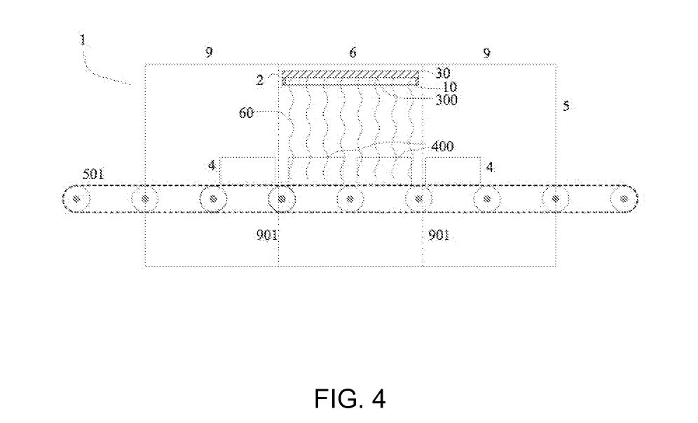

FIG. 4 is a diagram of the general architecture of an irradiator in accordance with embodiments of the present disclosure. The overall architecture of the panoramic irradiator is shown in this case with one panel 2 above the material being irradiated 4 as it is moved through irradiation section 6 by material transportation system 501. Enclosure 5 provides mechanical support for the system and is lined with shielding material, such as sheets of lead in thickness from 2 to 10 mm, to prevent X-ray flux from escaping into the surrounding area. Shielding sections 9, before and after irradiation section 6, prevent stray radiation from escaping the entrance and exits of irradiator 1, where people load and unload the material. The inner boundaries of the shielding section of the irradiator in FIG. 4 are shown by lines 901. The entrance and exits may also have doors or flaps or be made in contoured shapes to further prevent radiation from escaping. Material transport system 501, in this case a conveyor belt on rollers, is preferably made of material with either low attenuation of X-rays or material with high coefficients of X-ray reflection. Low atomic number Z materials have low attenuation and high

X-ray reflectance.

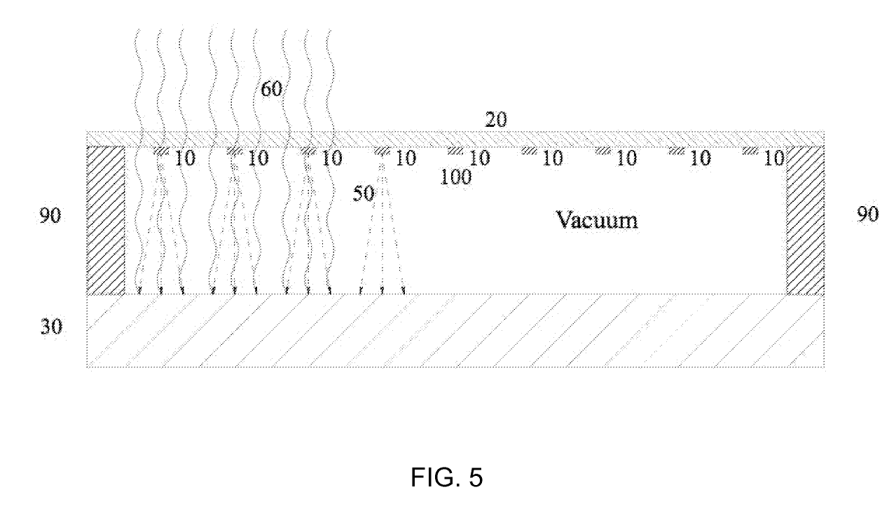

FIG. 5 is another diagram of the general architecture of flat panel X-ray sources in accordance with embodiments of the present disclosure. Detail as to a type of flat panel X-ray source which can be used in the irradiator of this disclosure is shown in FIG. 5. Source 2, the preferred flat X-ray source of this disclosure has an array 100 of cathodes 10 on exit window 20 of the source, with open space between the cathodes in the array so as to provide a wide area source of electrons. A wide, flat metallic X-ray target 30 is disposed opposite cathode array 100, the target having one major surface facing cathode array 100 and exposed to the vacuum of the source and the other major surface exposed to the exterior of the source. Exit window 20 and X-ray target 30 are the integral major parts of the vacuum enclosure of the source, with side walls 90 completing the vacuum enclosure. Cathode array 100 is operable to emit multiple electron beams 50 towards X-ray target 30 to generate X-ray flux 60, a portion of which will be emitted in the direction of cathode array 100 and pass through or by this array and out through exit window 20, and on to the material to be irradiated.

Exit window 20 of X-ray source 2 can be made of several different materials, including various types of glass, sapphire, ceramic, plastic that has been passivated for operation in vacuum, various forms of carbon sheet, beryllium and boron carbide. In general it is desirable for window 20 to be made of materials with a low atomic number Z and to be as thin as possible consistent with structural integrity under vacuum load, so as to allow as much of the X-ray flux as possible to pass through and be used for irradiation. Side walls 90 of the source can be made of the same materials as exit window 20. In general it is desirable to use the same materials for these parts of the source, or else materials that have a close match of thermal expansion, since heat from anode 30 propagates throughout the entire construction ands mismatched materials can cause stresses leading to vacuum leaks, rendering the source inoperable. Anode 30, which forms the X-ray target, can be made of any material, but is preferably made of a metal with a high Z number so as to increase X-ray generation. Common materials used for the anode in traditional X-ray tubes, such as tungsten, copper, molybdenum or ruthenium, can also be used for anode 30 in source 2. An exemplary materials set for these primary components of source 2 is a sapphire window, Macor or alumina side walls and an anode/target made of an 80/20 tungsten-copper alloy, all of which have a coefficient of thermal expansion in the neighborhood of 8.5 or 9×10⁻⁶ in./in.*/° C. Another exemplary materials set is soda lime glass for the window and side walls and plain tungsten for the anode, for matched coefficients of thermal expansion in the neighborhood of 4.5×10⁻⁶ in./in.*/° C. over the temperature range of interest. For anode 30, a flat sheet or slab of tungsten or tungsten-copper alloy of 1 mm or more in thickness will have more than sufficient rigidity to support the atmospheric load on the package, which is pumped down to an internal pressure of 10⁻⁵ to 10⁻⁸ Torr. Sheets of 3 to 6 mm have been used in prototypes and found to have good mechanical and thermal properties. Exemplary thicknesses for the side walls are 2 to 10 mm for glass or ceramic. Exit window 20 should be as thin as possible, preferably in the range of 0.5 to 10 mm for glass or ceramic, with the thinness of the window determined in part by the unsupported span over which it must maintain structural integrity under vacuum. Internal spacers, not shown in FIG. 5, can be used to reduce this span, with the spacers made of the same materials as the side walls or exit window.

The overall thickness of flat panel X-ray source 2 is determined by the thickness of window 20, the thickness of anode 30 and the wall and spacer separation between them. This separation will be considerably larger than the window and anode thicknesses, since sufficient distance must be provided between cathode array 100 and anode plate 30 to prevent arcs both inside the vacuum envelope of source 2 and between any externally exposed cathode and anode connections. Panel source 2 is operated at an anode to cathode voltage between 10 kV and 450 KV, with 80-200 KV being an exemplary range for medical product sterilization. In the 100 KV range for blood irradiation, a separation of 2 cm between cathode array 100 and anode 30 is more than sufficient to prevent vacuum breakdown and arcing inside the package. Externally, without additional electrical insulation and using prudent safety factors to account for humid air and other factors which can lead to the development of arcs, a separation of 15 cm or more is desirable. It is advantageous therefore to attach an oil, gas, vacuum or other insulation section to the externally exposed major surface of anode 30 so as to electrically isolate the anode from external arcs. This insulation section, such as an oil pan, is also used as or as part of a cooling system for anode 30, which allows source 20 to be operated at higher power levels.

Exemplary thicknesses of source 20 for operation up to 150 KV and with an insulation and cooling structure attached, are from 5 cm to 20 cm.

Cathode array 100 is formed directly on to, attached to or supported by window 20 of source 2. Array 100 may be made of either field emission cold cathodes or thermal filament cathodes. Space between the cathodes 10 of array 100 is provided to spread out the electron source generating the X-ray flux. This space can also be used for the placement of support structures for thermal filament cathodes or for resistors, buss lines and gating or extractor structures for field emission cold cathodes. Field emission cathode arrays are formed directly on window 20 using micro fabrication techniques. Alternatively, a field emission cathode array may be formed on a separate substrate which is then attached to or placed in front of flux exit window 20. Thermal filaments are stretched across the surface of window 20 and held in place by metallic, glass, ceramic or other support structures which are fused, frit sealed, welded or otherwise bonded to the window. Alternatively, a frame may be provided for the stretching and separation of thermal filament cathodes, and this frame may be attached to window 20 or placed in front of window 20 and supported by side walls 90.

In operation, the cathodes 10 in array 100 are caused to emit electrons, either through heating of the filament cathodes or through field emission extraction of current in cold cathode array. Hundreds of thousands or millions of cold cathodes can be formed into array 100, and in the case of thermal cathodes, numerous filaments can be stretched or patterned to make the array, so a very large number of electron beams will be emitted from array 100 and accelerated by the cathode to anode potential to hit anode 30, where they will generate X-rays across the surface of the anode through the classical Bremsstrahlung and characteristic line emission processes. X-ray flux in generated in all directions through these processes. About half of the generated X-rays will be emitted into anode/X-ray target 30 and serve no useful purpose. The other half will be emitted away from the anode and towards exit window 20 and the material to be irradiated, with some of the rays being absorbed by the side walls or internal spacers and some of the lower energy rays emitted in the direction of the target material being absorbed in array 100 or window 20. With a reasonably thin window 20, however, most of the X-ray flux that escapes the anode will be directed towards target material 4 and either be absorbed in the material, thereby serving the purpose of irradiation, or pass through material 4.

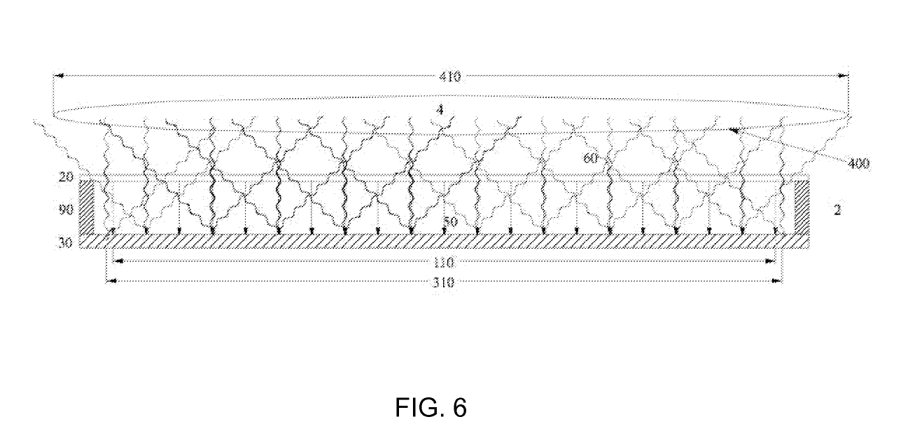

FIG. 6 is a diagram of the X-ray flux distribution from two flat panel X-ray source provided in accordance with embodiments of the present disclosure. This diagram shows the cross sections of the source provided in accordance with embodiments of the present disclosure and material being irradiated. Dimension 110 shows the width of the cross section of the cathode array on window 20, or by that part of array which is caused to emit electrons, while dimension 310 shows the cross sectional width of the flux generation area on anode 30 and dimension 410 shows the cross sectional width of surface 400, the proximate facing area of the material being irradiated 4. The flux generation area on anode 30, as indicated by cross sectional width 310, is essentially determined by the area of the cathode array on window 20, or by that part of array which is caused to emit electrons, as indicated by cross sectional width 110. This is because at high anode potential, and without any means of deliberately deflecting electron beams 50, these beams will head straight at anode 30 and diverge laterally by only a very small distance. Only those beams produced by cathodes at the outer perimeter of the emitting area of array 100 will fall outside of the corresponding area on the anode, and this by a very slight degree. Most of the X-rays 60 which are generated on anode 30 will in turn be directed towards the corresponding area 400, over its cross sectional width 410, on the proximate surface of the material being irradiated 4. Some of the X-rays, particularly those emitted around the perimeter of the anode, will be absorbed in the side walls, and a small percentage will be emitted at such a shallow angle as to cause them to miss irradiation target surface 400, but with a wide flux generation area, substantially all of the X-ray flux leaving anode 30 will be directed towards proximate surface 400 on the material to be irradiated.

The wide area of anode 30 provides one of the major advantages of source 2, which is relatively easy thermal management of the heat generated on the anode, since the heat can be dissipated over a broad area and the exterior side of anode 30 can be directly coupled to atmosphere, forced air, oil bath or circulating fluid heat dissipation systems.

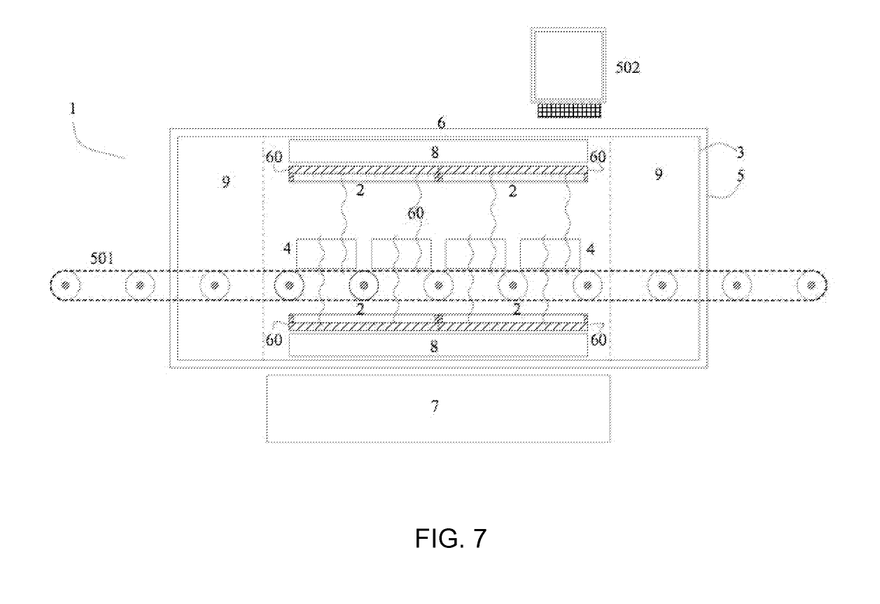

FIG. 7 is a diagram of another embodiment of an irradiator in accordance with embodiments of the present disclosure. Further aspects of irradiator 1 are shown in FIG. 7, in this case with multiple flat panel X-ray sources 2 arranged at the top and bottom of irradiation section 6 of enclosure or frame 5, with anodes 30 closest to the enclosure and windows 20, with the cathode arrays, facing inwards. Tiling the flat panel X-ray source together, which can done both along the axis of movement of the material to be irradiated and in the transverse direction, will provide a larger flux generation area. Numerous panels can be tiled together along the axis of movement of the material, for a very long irradiator. The irradiators themselves may also be made modular so that more than one can be attached end to end, and share a common material transport system, so as to further lengthen the flux generation area. The flat panel X-ray source can be activated via the irradiator control system to deliver radiation doses matched to the material to be irradiated. For example, with material having a large proximate surface area, all the panels on each side can be activated. For smaller doses, a smaller number of panels may be activated, to provide for an efficient use of power. A further advantage of tiling several flat panel X-ray sources together on a side of an irradiator is redundancy, since if one panel fails the other can still be operated.

As material 4 is transported through irradiation section 6 it receives X-ray flux 60 from above and below from panels 2. Material transport system 501 may be a conveyor belt using rollers, as shown in FIG. 7, a hook and gantry systems, pipes for transporting fluids, separately powered trucks or carts, or any other system which can move the material through the irradiator. A means for rotating the material as it is transported through the irradiation section may also be incorporated to provide a more uniform radiation dose in the material. Power supply 7 can be either internal to enclosure 5 or external. It will preferably incorporate a voltage amplifier to bring municipal power up to the high potential needed for X-ray generation, although it may also comprise a relay system for delivering current to the irradiator from high voltage transmission lines. Power supply 7 may also incorporate a generator to produce its own electricity from fuel or another source of power.

Enclosure 5 is lined with shielding material 3, such as lead sheet, to absorb any radiation which is not absorbed by material 4 or opposing anodes 30 of the flat panel X-ray sources. Heat exchanger system 8 may be provided to remove heat from the flat panel X-ray source anodes during high power operation. The heat exchanger may be directly attached to the anode or may be displaced from the anode. Flat panel X-ray sources 2 may have an oil-filled casing attached to cover anodes 30 and provide high voltage insulation. The oil can be circulated through tubings to a displaced heat exchanger 8 to allow operation at high power levels. The heat exchanger system may incorporate fans, baffles or other means to dissipate heat to outside the irradiator. Heat exchanger system 8 may be enclosed fully or partially by enclosure 5. Other high voltage insulation, such as plastic or ceramic sheets, may be used in place of an oil casing for the X-ray source anodes. In this case, types of heat exchanger systems may be used, such as forced air passed over the high voltage insulation and the X-ray source, separate pumped water o oil cooling or gas insulation. Thermal insulation structures may be built into material transport system 501 to isolate the material from the heat generated during X-ray production. Doors, flaps of lead sheet or serpentine shaped channels may be used at the entrance and exit of the irradiator as an additional means of keeping radiation from escaping the irradiator. Interlocks and other safety features may be incorporated for safer operation. Interlocks on a door, for example, will shut off power when the door is opened. An X-ray ON light on the outside of the box may be activated when power is supplied to the X-ray sources. An emergency switch may be provided to turn off power in case of emergency. Controls may provided to set the irradiation time, current levels and voltage to the X-ray sources. A bar code scanner may also be attached to the irradiator to allow tracking of throughput. An internal radiation dose measurement system may be provided for recording the dose delivered to each lot of material irradiated. All of these control, emergency and tracking features may be operated separately, in combination, or in one embodiment by control system 502, a computing device which may be directly connected to the irradiator and provide a use-design face such as a touch screen that allows the user-operator to control all functions of the irradiator. Additionally, a computer for controlling one or more of the functions of the irradiator may connect the system to a local network for remote operation and data savings.

The size of irradiator 1 is determined primarily by its intended use. Small, desktop systems may be used, for example, to provide sterilization or other types of radiation processing to medical devices, human blood supplies, contact lenses or pharmaceuticals. Floor-standing models in the size range of airport baggage scanners or larger may be used, for example, to sterilize large quantities of medical products inside a factory or factory shipping bay, or mail inside a sorting facility. Larger systems can be used for bulk quantities of medical products or foodstuffs, or example. The irradiator may be stationary or mobile. For example, even a large irradiator can be placed onto a truck trailer or into a large (e.g. 40′ long) shipping container and transported to a point of production, transshipment or distribution to reduce shipping and handling costs associated with irradiation.

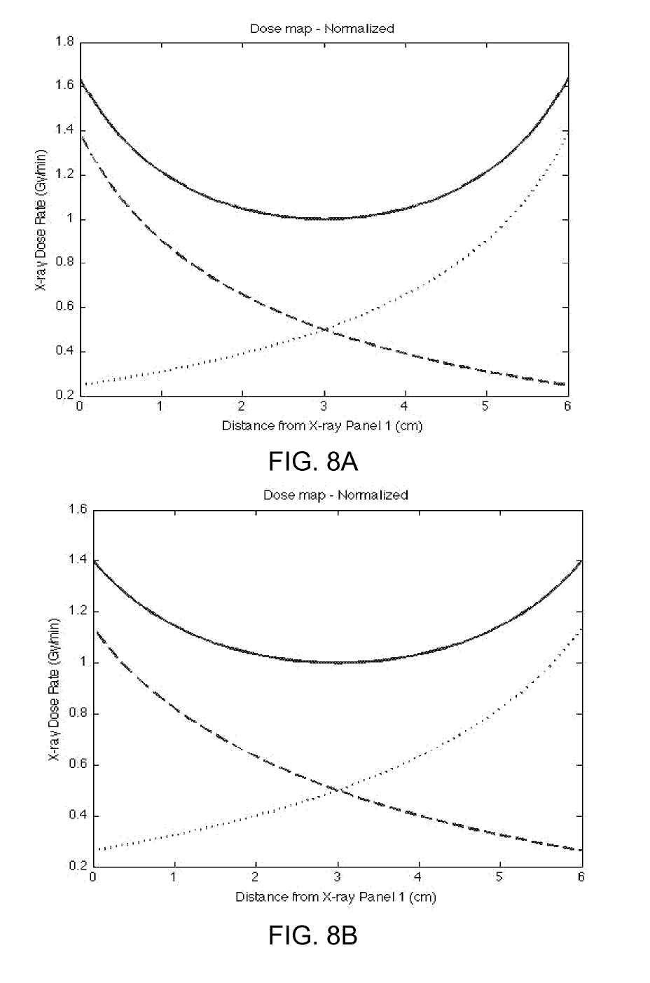

FIGS. 8A and 8B shows calculated dose-depth maps of X-ray flux delivered to material in an irradiator of the present disclosure having flat panel X-ray sources placed on opposite sides of the material. In this case, the material to be irradiated is blood contained in blood bags. The normalized X-ray dose rate in Gy/min is plotted as a function of the distance from the X-ray sources. The dashed lines show the dose rate as a function of the distance from one X-ray source and the dotted lines show the dose rate as a function of the distance from the other X-ray source. The solid lines are the combined dose rate from both sources as a function of the distance. The plots are shown for 100 kV and 150 kV operating voltage. As shown in FIG. 8, dose uniformity is substantially improved by irradiating the material from opposite sides.

It will be appreciated from FIG. 5 that if all the cathodes in the cathode array of source 2 were evenly distributed over the source exit window and all operated at the same current level, the X-ray flux would be highest from the middle of the source, owing to the greater overlap of X-ray flux generation sites at the center of anode 30 as compared to the sides. Further embodiments of the disclosure provide for even flux distribution across the panel or panels as they are used on a flux generating side of the irradiator.

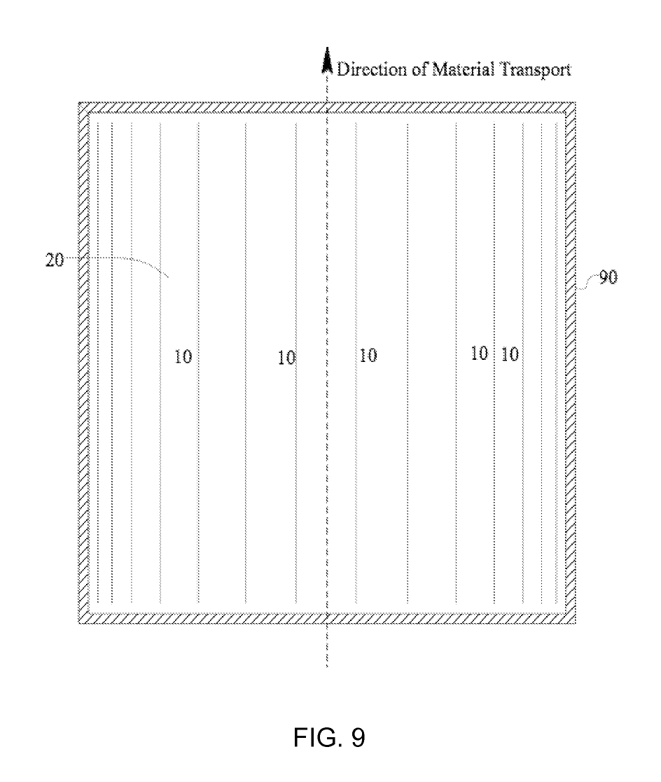

FIG. 9 shows an embodiment of the present disclosure in which the cathodes in the array of a flat panel X-ray source are made denser towards the edges of the array away from the center, thereby smoothing out the flux distribution of the source across its emitting area. Here, the cathodes on array 100 on window 20 are made denser towards the edges of the array near source walls 90 and sparser towards the center, as defined by the axis of movement of the material being irradiated. This provides for a corresponding change in the density of X-ray flux generation on the anode. In the case of thermal filament cathodes, the filaments are spaced closer together closer to the edge of the array. In the case of cold cathodes, the areal density the individual emitters can be increased closer to the edge of the array. For example, a cold cathode array used in a flat panel X-ray source might have an average of 24,000 individual cathodes per square centimeter, but the density of the cathode at the center of the array could be only 5,000/cm², while the density at the edges of the array could be over 50,000/cm². In another embodiment of the disclosure, the cathodes in the array may be supplied with increasingly higher current as they get closer to the edge of the array, as defined by the axis of movement of the material being irradiated.

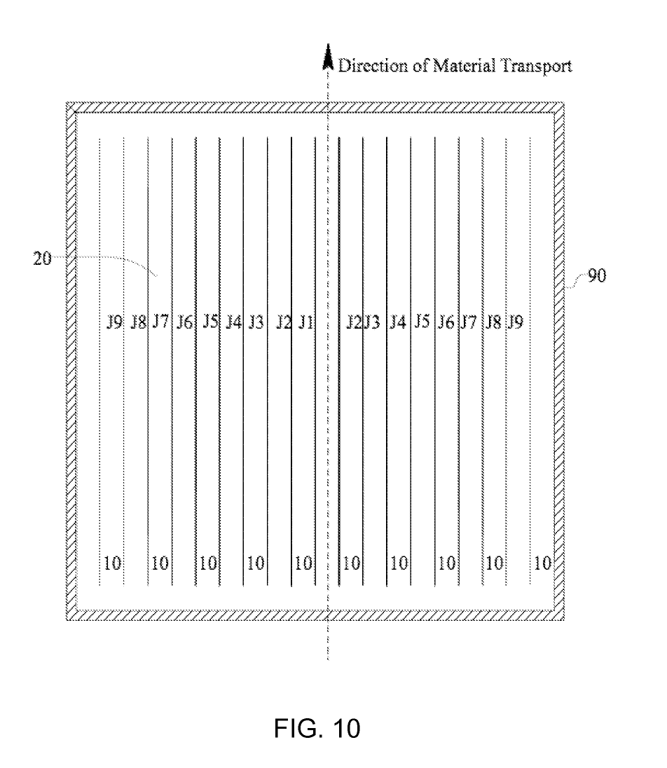

FIG. 10 shows an embodiment of the present disclosure in which the cathodes of the array in a flat panel X-ray source are supplied with greater current the further the cathodes are away from the center of the array and towards the edges of the array, thereby smoothing out the flux distribution of the source across its emitting area. J1-J9 indicate increasingly higher current levels, these increasing levels can be done with an array of evenly dense emitters, in addition to the array shown in FIG. 9 where the density is higher towards the edges of the array. In a tiled flat panel X-ray source configuration, such as that shown in FIG. 7, but with more than four panels, variable cathode density or current density can be supplied to different panels, to smooth out X-ray flux density from the entire flux generation area.

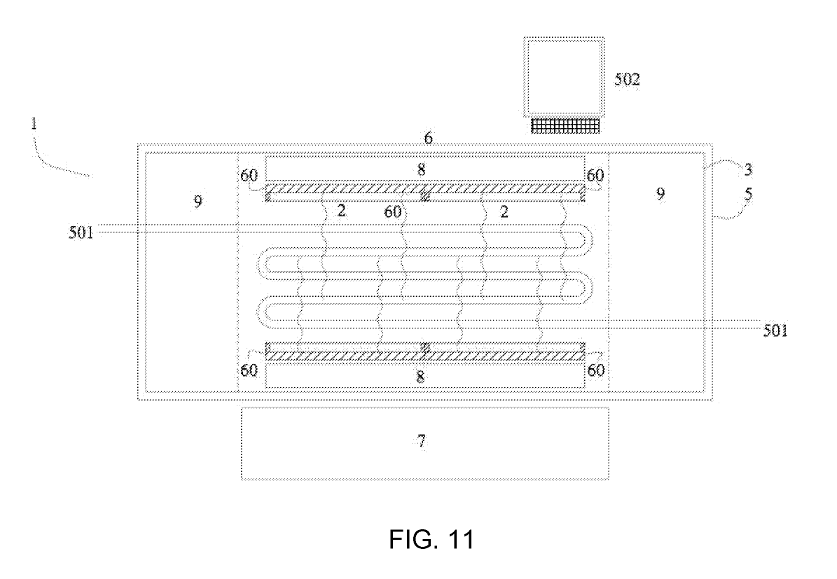

FIG. 11 is a diagram that shows one of the several alternative material transport systems 501 that can be used in the irradiator in accordance with embodiments of the present disclosure. In this embodiment a serpentine configuration of pipes transports fluids. Pumps may be provided on the entrance or exit sides of the irradiator to move the fluids. Other configurations of pipes may be similarly used, such as coils, horizontally disposed serpentine or racks of serpentine piping. Low Z materials are preferred for the pipes so as to allow more of the X-ray flux to reach the fluid.

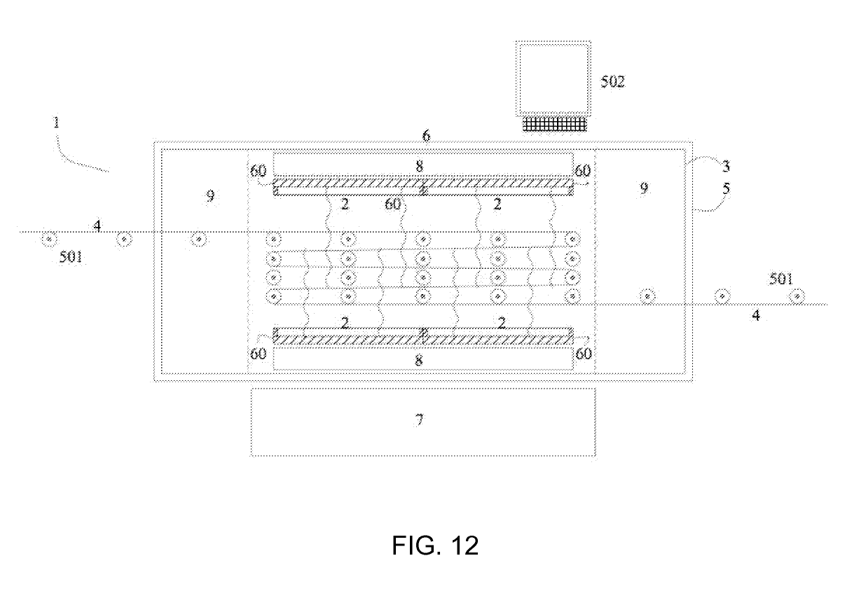

FIG. 12 shows another material transport system 502, in this embodiment a roller configuration which allows flexible sheets of material to be moved through the irradiator section 6. This configuration may be used in medical, food or other packaging or fabrication applications requiring sterile materials. In all these embodiments, including the conveyor belt system shown in FIG. 4, the position of the flat panel X-ray sources 2 or parts of the material transport system 5, such as the rollers shown in FIG. 12, may be made adjustable so as to bring the X-ray source closer to or further away from the materials being irradiated, or to increase or decrease the amount the material passing through irradiation section 6. One effect of this adjustment will be to vary the amount of ozone being generated during irradiation by ionization of O₂ in the air contained in the irradiator. This ozone can be a useful byproduct of the irradiation process in some applications, such as sterilization.

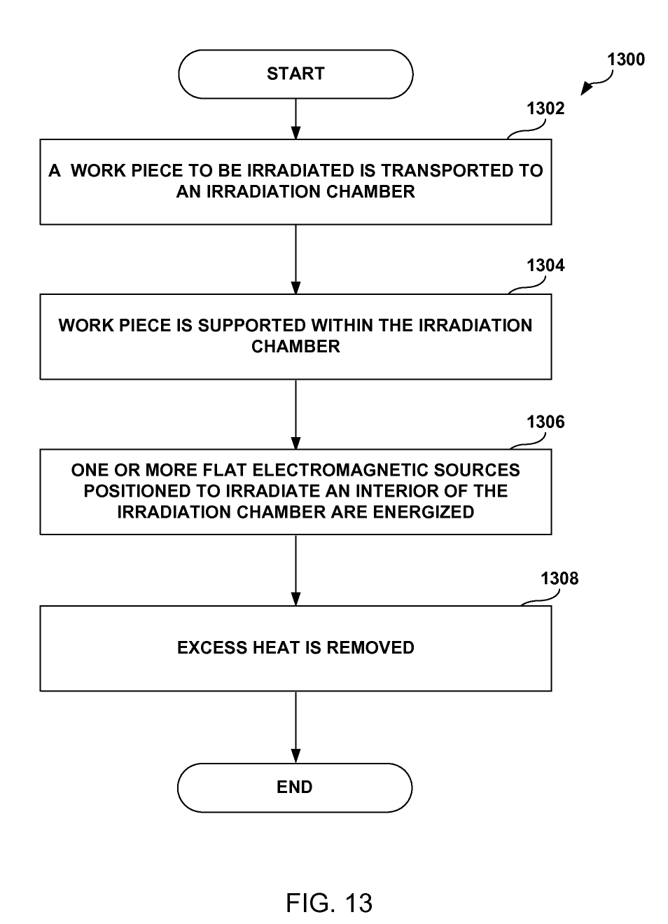

FIG. 13 provides a logic flow diagram of a method of irradiating materials in accordance with embodiments of the present disclosure. Operations 1300 begin with block 1302 where a work piece to be irradiated is transported to an irradiation chamber. This may involve placing materials directing within a chamber through a shielded portal that allows access as discussed with reference to the prior FIGs., placing the materials on a conveyor or transport system as discussed with reference to FIGS. 4, 7 and 12, or pumping fluids through the chamber as discussed with reference to FIG. 11. A carousel within the irradiation chamber may be used to rotate the work piece within the irradiation chamber for uniform distribution of the electromagnetic flux to the work piece. In block 1304, the work piece is supported within the irradiation chamber with a low attenuation support mechanism. Then, in block 1306, one or more flat electromagnetic sources positioned to irradiate an interior of the irradiation chamber are energized at a controlled energy level and time. Excess heat is removed from the one or more flat electromagnetic source with a heat transfer system in Block 1308. The exterior is shielded from the electromagnetic flux within the irradiation chamber by a shielding system. The electromagnetic flux comprising an X-ray flux or an ultraviolet flux. A process controller may be used to coordinates the operation of the irradiation chamber; one or more flat electromagnetic sources, the heat transfer system; and the interlock system.

In summary, the present disclosure provides an apparatus and method for the X-ray irradiation of materials. This apparatus includes an irradiation chamber, a number of flat electromagnetic (X-ray) sources, a support mechanism, a heat transfer system, and a shielding system. A shielded portal within the shielding system allows access to an interior volume of the irradiation chamber. The shielded portal allows materials to be placed in and withdrawn from the irradiation chamber. When closed, the shielded portal allows a continuous shielded boundary of the interior volume of the irradiation chamber. The electromagnetic sources are positioned on or embedded with interior surfaces of the irradiation chamber. These electromagnetic sources may generate an electromagnetic flux, such as an X-ray flux, where this flux is used to irradiate the interior volume of the irradiation chamber and any materials placed therein. The materials placed within the interior of the chamber may be supported by a low attenuation support mechanism. This low attenuation support mechanism does not substantially reduce the X-ray flux intended to irradiate the materials placed within the interior volume of the irradiation chamber. Additionally the irradiation chamber may have a heat transfer system thermally coupled to the irradiation chamber and electromagnetic sources in order to remove heat from the interior surfaces of the irradiation chamber. The shielding system external to the irradiation chamber prevents unwanted radiation from escaping from within the irradiation chamber.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

What is claimed is:

1. A panoramic irradiator comprising:
a shielded enclosure;
at least one planar X-ray source within the shielded enclosure, the at least one X-ray planar source operable to emit an X-ray flux across an area substantially equal to the proximate facing surface area of material transportable through an irradiation section of the panoramic irradiator by a material transport system, the material transport system operable to be loaded and unloaded outside the panoramic irradiator.

2. The irradiator of claim 1 in which the at least one X-ray sources are flat panel X-ray sources comprise:
a cathode array formed on a flux exit window of the at least one planar X-ray source; and
a wide, flat metallic X-ray target disposed opposite the cathode array, the wide, flat metallic X-ray target comprising:
a first major surface facing the cathode array and exposed to the vacuum of the source; and
a second major surface exposed to an exterior of the source, the exit window and X-ray target being integral major parts of a vacuum enclosure of the source; and
the cathode array operable to emit multiple electron beams towards the X-ray target to generate the X-ray flux, a portion of the X-ray flux emitted in a direction of the cathode array, passing by or through the cathodes and out the exit window.

3. The irradiator of claim 2, the cathode array of the flat panel X-ray source comprises a cold cathode array with open space between the cathodes in the array.

4. The irradiator of claim 2, the cathode array of the flat panel X-ray source comprises a thermal filament array with open space between the filaments.

5. The irradiator of claim 1, the material to be irradiated comprises at least one product selected from the group consisting of:
medical products;
bulk solids;
grains;
intermediate materials used during a manufacturing process;
food;
mail;
packages;
fluids;
water;
wastewater,
blood products;
wine;
industrial wastes; and
medical wastes.

6. The irradiator of claim 1, the at least one X-ray planar source comprises a first X-ray sources and a second X-ray source, the first X-ray sources and second X-ray source disposed on opposite sides of the irradiator enclosure.

7. The irradiator of claim 2, the at least one X-ray planar source comprises a plurality X-ray planar sources tiled together on a side of the irradiator enclosure.

8. The irradiator of claim 2, a density of the cathodes in the cathode array of the at least one X-ray planar source is varied to provide a substantially even distribution of X-ray flux from the anode.

9. The irradiator of claim 2 in which the current supplied to the cathodes in the cathode array of the flat panel X-ray source is varied to provide even distribution of the X-ray flux from the anode.

10. The irradiator of claim 1 further comprising a process controller operable to coordinate the operation of:
the irradiation section of the irradiator;
the at least one X-ray source;
a heat transfer system operable to remove heat from the X-ray source; and
an interlock system operable to shut off power to the X-ray sources in the event the material transport system is not in service, X-rays are leaking from the enclosure or high voltage electrical current has deviated from its intended circuit.

11. A system comprising:
an irradiation chamber;
at least one substantially planar X-ray source positioned to irradiate an interior of the irradiation chamber;
a transport mechanism operable to transport a work piece to be irradiated to and from the irradiation chamber;
a low attenuation support mechanism operable to support a work piece to be irradiated within the irradiation chamber;
a shielding system placed on the exterior surfaces of the irradiation chamber to prevent inadvertent irradiation outside of the irradiation chamber; and
shielded protection covers that cover the transport mechanism and substantially shield the exterior environment from radiation flux that escapes the irradiation chamber.

12. The system of claim 11, the at least one substantially planar X-ray source comprising:
a hermetically sealed volume;
a large area cathode operable to emit electrons (e⁻), the large area cathode forming an outer surface of the hermetically sealed volume;
a large area anode, the anode within the hermetically sealed volume, the anode and cathode are substantially parallel, and the area of the cathode and the area of the anode are substantially equal;
the anode operable to generate an X-ray flux substantially normal to a large area surface of the anode in response to the e^{-,}s impacting the anode;
the cathode substantially transparent to the X-ray flux, the X-ray flux exiting the hermetically sealed volume through the cathode and into the interior volume of the irradiation chamber.

13. The system of claim 12, further comprising a shielded portal to allow access to the irradiation chamber.

14. The system of claim 13, further comprising an interlock system coupled to the shielded portal and the at least one substantially planar X-ray source, the interlock system operable to prevent irradiation of the irradiation chamber when the shielded portal is open.

15. The system of claim 12, further comprising a process controller operable to coordinate the operation of:
the irradiation chamber;
the at least one substantially planar X-ray source;
the heat transfer system; and
the interlock system.

16. A method comprising:
transporting a work piece to be irradiated to and from an irradiation chamber with a transport mechanism;
supporting the work piece within the irradiation chamber with a low attenuation support mechanism;
energizing at least one substantially planar X-ray source positioned to irradiate an interior of the irradiation chamber;
irradiating the work piece within the irradiation chamber;
removing excess heat from the at least one flat electromagnetic source with a heat transfer system; and
shielding the exterior from the electromagnetic flux within the irradiation chamber with shielded protection covers operable to cover the transport mechanism and substantially shield the exterior environment from radiation flux that escapes the irradiation chamber.

17. The method of claim 16, wherein a shielded portal allows access to the irradiation chamber.

18. The method of claim 16, wherein a carousel within the irradiation chamber, rotates the work piece within the irradiation chamber for uniform distribution of the X-ray flux to the work piece.

19. The method of claim 16, wherein a process controller operable to coordinates the operation of:
the irradiation chamber;
the at least one substantially planar X-ray source;
the heat transfer system; and
the interlock system.

20. The method of claim 16, wherein a plurality of substantially planar X-ray sources is tiled to irradiate the irradiation chamber, the tiled substantially planar X-ray sources individually or simultaneously.

21. A system comprising:
an irradiation chamber;
at least one substantially planar X-ray source positioned to irradiate an interior of the irradiation chamber;
a transport mechanism operable to transport a work piece to and from the irradiation chamber;
a low attenuation support mechanism operable to support a work piece to be irradiated within the irradiation chamber;
a heat transfer system operable to remove heat from the at least one substantially planar X-ray source;
a shielding system placed on the exterior surfaces of the irradiation chamber to prevent inadvertent irradiation outside of the irradiation chamber; and
a process controller operable to coordinates the operation of:
the irradiation chamber;
the at least one substantially planar X-ray source;
the heat transfer system; and
the interlock system.
